(19) United States
(12) Patent Application Publication
(10) Pub. No.: US 2008/0051796 A1
(43) Pub. Date: Feb. 28, 2008

(54) Graft retention tack

(75) Inventors: Jeffrey H. Nycz, Collierville, TN (US); Michael R. Burns, Memphis, TN (US); Stanley W. Olson, Germantown, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: WARSAW ORTHOPEDIC, INC., Warsaw, IN (US)

(21) Appl. No.: 11/496,861

(22) Filed: Aug. 1, 2006

(51) Int. Cl. A61F 5/00

(52) U.S. Cl. 606/86

(57) ABSTRACT

A graft retention tack is disclosed and can include a post configured to receive a bone growth factor and a head configured to receive a cartilage growth factor.

### FIELD OF THE DISCLOSURE

The present disclosure relates generally to devices for repairing diarthrodial joints. More specifically, the present disclosure relates to repairing articular cartilage.

### BACKGROUND

A human body includes numerous joints. One type of joint includes diarthrodial joints. Diarthrodial joints, for example, include knee joints, hip joints, shoulder joint, wrist joints, finger joints, thumb joints, and elbow joints. Each diarthrodial joint can include articular cartilage. Articular cartilage is a type of hyaline cartilage that is a smooth, glistening white tissue that lines the surfaces of opposing bones in diarthrodial joints.

Articular cartilage facilitates the movement of one bone against another. Articular cartilage has a relatively low coefficient of friction and a relatively high compressive strength. As such, articular cartilage is ideally suited for placement in joints, such as the knee and hip. Loss of articular cartilage from the ends of bones forming a diarthrodial joint can be a source of profound pain and disability. Further, the loss of articular cartilage can eventually lead to complete degeneration of a joint and may necessitate replacement of the joint.

Accordingly, there is a need for a device for repairing and regenerating articular cartilage.

### BRIEF DESCRIPTION OF THE DRAWINGS

FIG. 1 is an exploded lateral plan view of a first embodiment of a graft retention tack;

FIG. 2 is a cross-section exploded view of the first embodiment of the graft retention tack;

FIG. 3 is a superior plan view of the first embodiment of the graft retention tack with a graft containment cap removed;

FIG. 4 is an inferior plan view of the first embodiment of the graft retention tack with the graft containment cap removed;

FIG. 5 is a superior plan view of the graft containment cap;

FIG. 6 is an inferior plan view of the graft containment cap;

FIG. 7 is a cross-section view of the graft containment cap taken along line 7-7 in FIG. 6;

FIG. 8 is a lateral plan view of the first embodiment of the graft retention tack with the graft containment cap installed;

FIG. 9 is a cross-section view of the first embodiment of the graft retention tack with the graft containment cap installed;

FIG. 10 is a superior plan view of the first embodiment of the graft retention tack with the graft containment cap installed;

FIG. 11 is a flow chart illustrating a first method of using a graft retention tack;

FIG. 12 is an exploded lateral plan view of a second embodiment of a graft retention tack;

FIG. 13 is a cross-section exploded view of the second embodiment of the graft retention tack;

FIG. 14 is a superior plan view of the second embodiment of the graft retention tack with an interior graft containment cap and an exterior graft containment cap removed;

FIG. 15 is an inferior plan view of the second embodiment of the graft retention tack with the interior graft containment cap and the exterior graft containment removed;

FIG. 16 is a superior plan view of the exterior graft containment cap;

FIG. 17 is an inferior plan view of the exterior graft containment cap;

FIG. 18 is a cross-section view of the exterior graft containment taken along line 18-18 in FIG. 17;

FIG. 19 is a superior plan view of the interior graft containment cap;

FIG. 20 is an inferior plan view of the interior graft containment cap;

FIG. 21 is a cross-section view of the interior graft containment cap taken along line 21-21 in FIG. 19;

FIG. 22 is a lateral plan view of the second embodiment of the graft retention tack with the interior graft containment cap and the exterior graft containment cap installed;

FIG. 23 is a cross-section view of the second embodiment of the graft retention tack with the interior graft containment cap and the exterior graft containment cap installed;

FIG. 24 is a superior plan view of the second embodiment of the graft retention tack with the interior graft containment cap and the exterior graft containment cap installed; and

FIG. 25 is a flow chart illustrating a second method of using a graft retention tack.

### DETAILED DESCRIPTION OF THE DRAWINGS

A graft retention tack is disclosed and can include a post configured to receive a bone growth factor and a head configured to receive a cartilage growth factor.

In another embodiment, a graft retention tack is disclosed and can include a post having a proximal end and a distal end. The post can receive a first carrier. The graft retention tack can also include a head attached to the distal end of the post. The head can receive a second carrier.

In yet another embodiment, a method of treating a patient using a graft retention tack having a post and a head attached to the post is disclosed. The method can include wetting a first carrier with a first growth factor and inserting the first carrier into a post of the graft retention tack. Further, the method can include wetting a second carrier with a second growth factor and inserting the second carrier into a head of the graft retention tack.

In still another embodiment, a method of treating a patient using a graft retention tack having a post and a head attached to the post is disclosed. The method can include wetting a first carrier with a first growth factor and inserting the first carrier into a post of the graft retention tack. Moreover, the method can include engaging an interior graft retention cap with the post to substantially seal the post.

In yet still another embodiment, a method of treating a patient using a graft retention tack having a post and a head attached to the post is disclosed. The method can include inserting a first carrier into a post of the graft retention tack and inserting a second carrier into a head of the graft retention tack.

### Description of a First Embodiment of a Graft Retention Tack

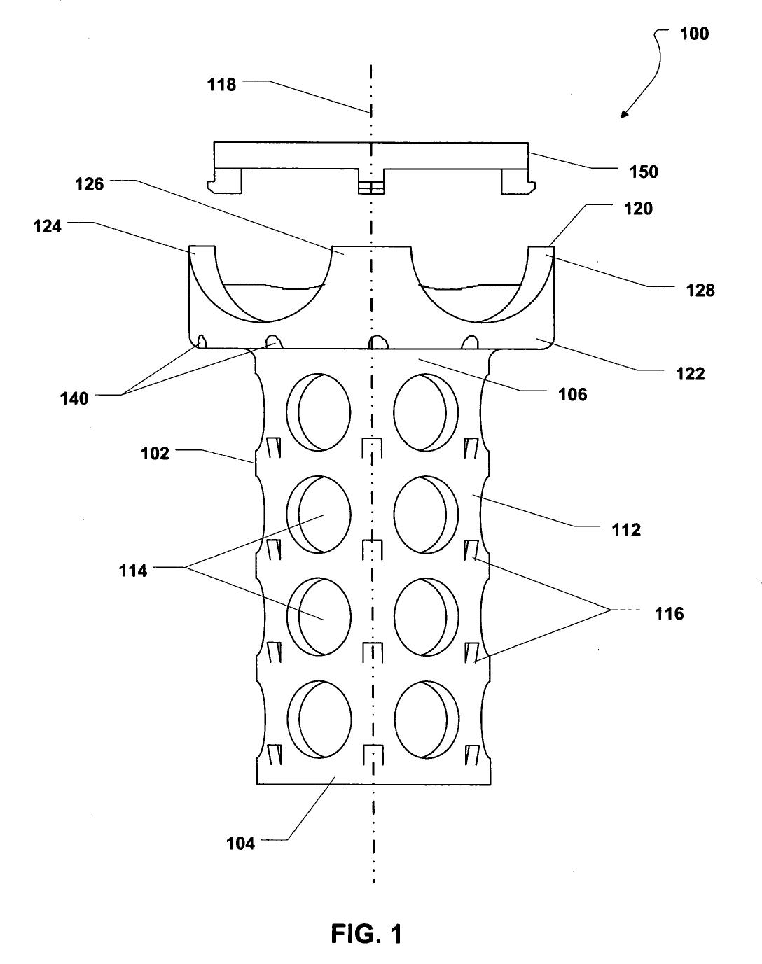

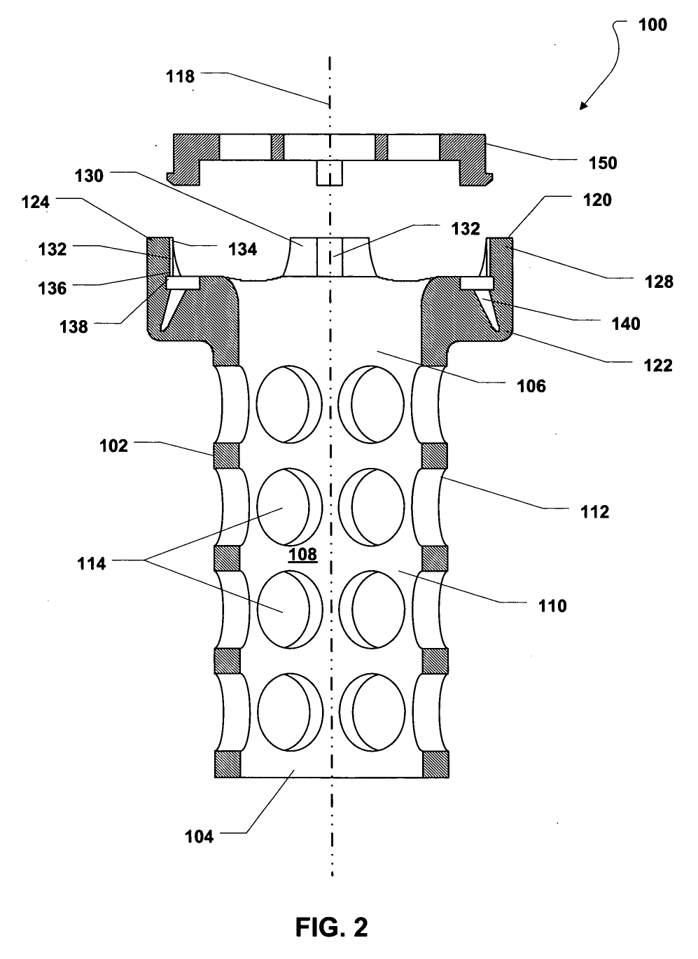

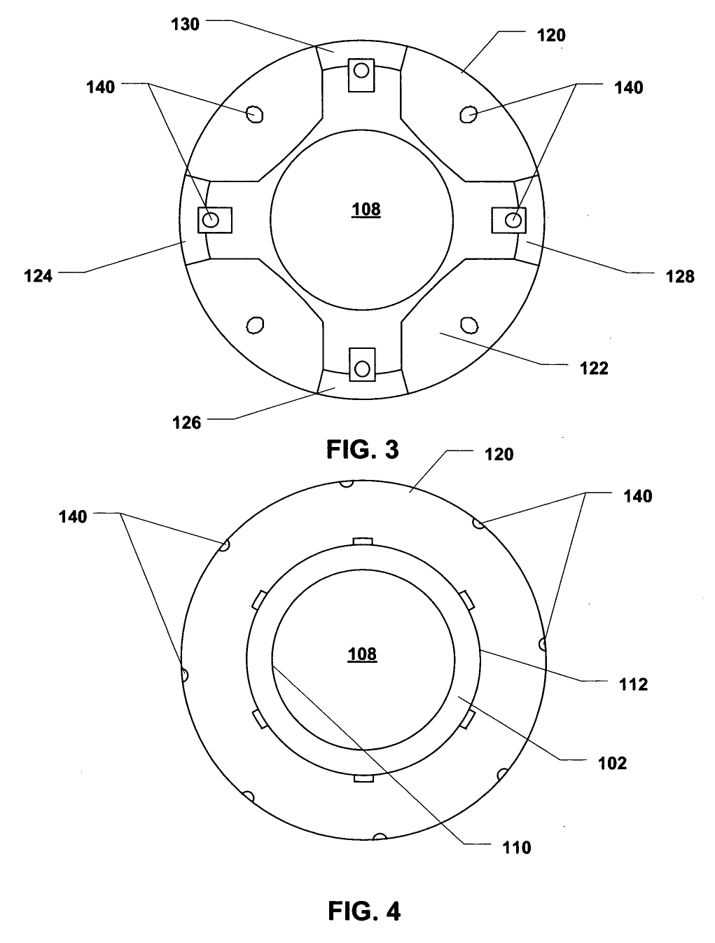

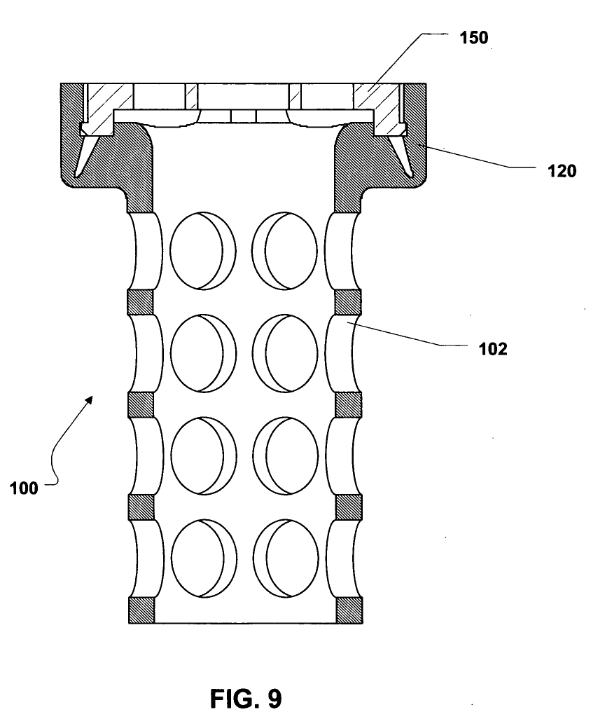

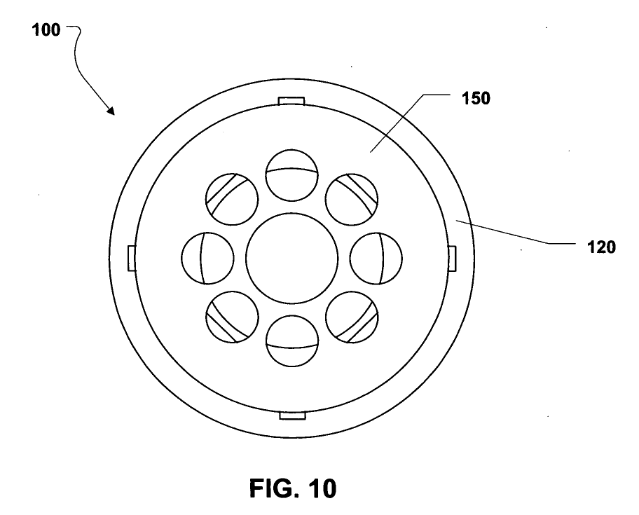

Referring to FIG. 1 through FIG. 9, a graft retention tack is shown and is generally designated 100. As illustrated in FIG. 1 and FIG. 2, the graft retention tack 100 can include a post 102. The post 102 can include a proximal end 104 and a distal end 106. In a particular embodiment, the post 102 can be generally cylindrical. Further, the post 102 can be generally hollow and can include an interior chamber 108. The post 102 can also include an interior surface 110 and an exterior surface 112.

In a particular embodiment, the post 102 of the graft retention tack 100 can include a plurality of fenestrations 114 formed in the post 102 and leading from the interior chamber 108 of the post 102. Further, the post 102 can include a plurality of tissue engagement structures 116 that can extend radially from the exterior surface 112 of the body 102. The post 102 can also define a longitudinal axis 118. In an alternative embodiment, the post 102 can be porous and the porosity of the post can allow a bone growth factor, described in detail below, to seep from the interior chamber 108 of the post 102. Further, the post 102 itself can be dosed, or wetted, with a bone growth factor such that the bone growth factor is introduced into the pores of the post 102. After installation, the bone growth factor can seep from the pores of the post 102 of the graft retention tack 100.

FIG. 1 and FIG. 2 further indicate that a head 120 can be attached to the distal end 106 of the post 102. The head 120 can be generally cylindrical and can include a base 122 that can be oriented substantially perpendicular to the post 102. A first cap engagement structure 124 can extend substantially perpendicular from the base 122 of the head. A second cap engagement structure 126 can extend substantially perpendicular from the base 122 of the head. A third cap engagement structure 128 can extend substantially perpendicular from the base 122 of the head. Moreover, a fourth cap engagement structure 130 can extend substantially perpendicular from the base 122 of the head 120. Each cap engagement structure 124, 126, 128, 130 can be substantially parallel to the longitudinal axis 118 of the post 102.

As illustrated in FIG. 2, each cap engagement structure 124, 126, 128, 130 can include a slot 132. Each slot 132 can include a proximal end 134 and a distal end 136. Further, the proximal end 134 of each slot 132 can be formed with a detent 138. As described in detail below, the detent 138 of each slot 132 can be configured to receive and engage a locking arm from a graft containment cap.

FIG. 2 through FIG. 4 further show that the head 120 of the graft retention tack 100 can include a plurality of holes 140 therethrough. In a particular embodiment, the holes 140 can be formed in the base 122 of the head 120. Further, the holes 140 can be equally spaced radially around the base 122 of the head 120. Also, the holes 140 can be formed at an angle through the base 122 of the head 120.

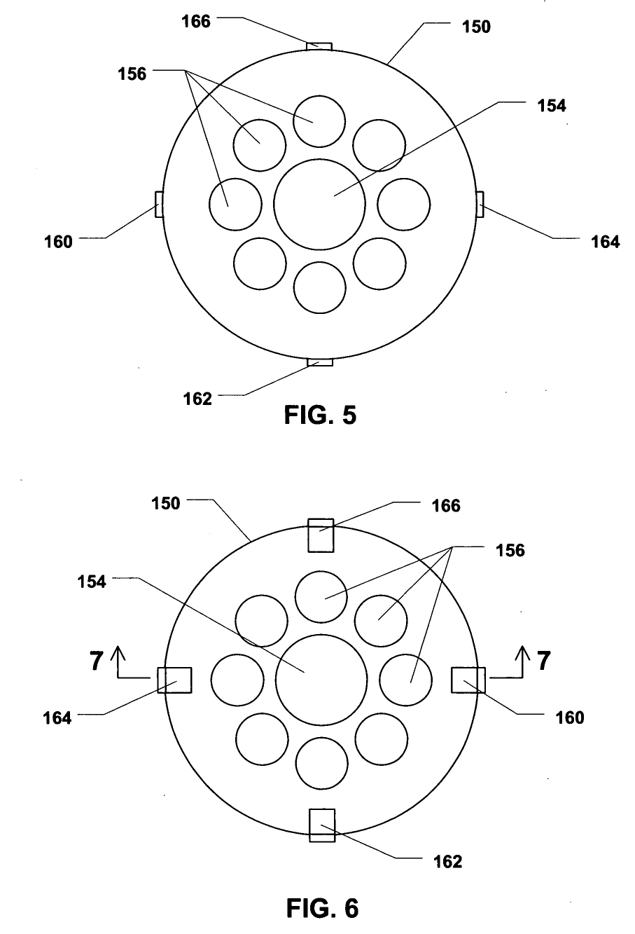

As shown in FIG. 1 and FIG. 2, the graft retention tack 100 can further include a graft containment cap 150. As illustrated in FIG. 5 through FIG. 7, the graft containment cap 150 can include a base 152. In a particular embodiment, the base 152 can be a generally cylindrical disc. The base 152 can be formed with a central hole 154 therethrough. Further, the base 152 can be formed with a plurality of radial holes 156 that can be equally spaced radially around the central hole 154 within the base 152.

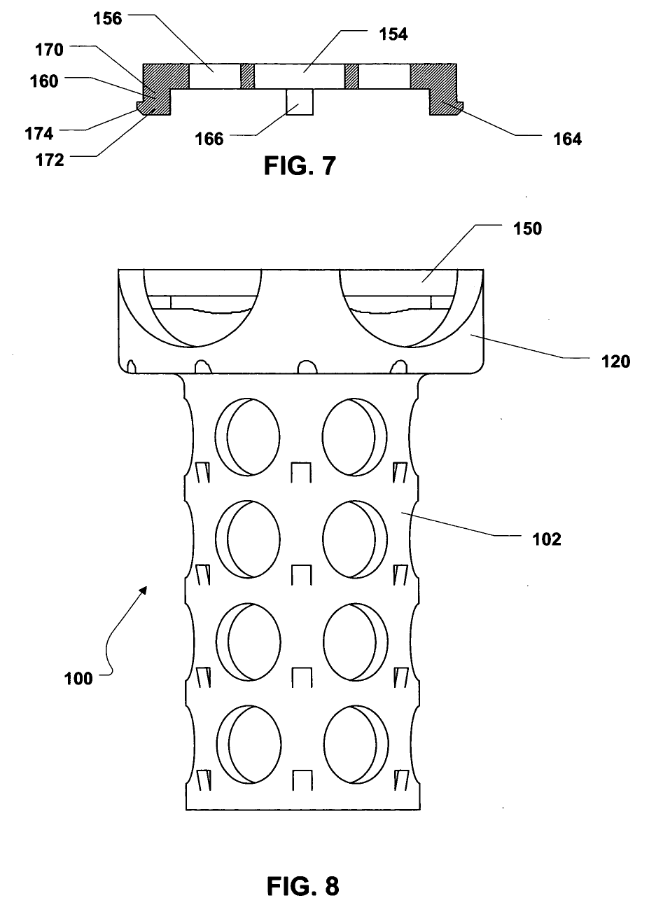

FIG. 5 and FIG. 7 indicate that the graft containment cap 150 can include a first locking arm 160 that can extend substantially perpendicular from the base 152 of the graft containment cap 150. A second locking arm 162 can extend substantially perpendicular from the base 152 of the graft containment cap 150. A third locking arm 164 can also extend substantially perpendicular from the base 152 of the graft containment cap 150. Further, a fourth locking arm 166 can extend substantially perpendicular from the base 152 of the graft containment cap 150.

As illustrated in FIG. 6, each locking arm 160, 162, 164, 166 can include a proximal end 170 and a distal end 172. Further, a tooth 174 can extend from the distal end 172 of each locking arm 160, 162, 164, 166. In a particular embodiment, the graft containment cap 150 can fit into the head 120 of the graft retention tack 100. Further, each locking arm 160, 162, 164, 166 can fit into a cap engagement structure 124, 126, 128, 130. In particular, each locking arm 160, 162, 164, 166 can fit into a slot 132 formed in a cap engagement structure 124, 126, 128, 130. Moreover, the tooth 174 that extends from each locking arm 160, 162, 164, 166 can engage the detent 138 formed in the slot 132 of each cap engagement structure 124, 126, 128, 130.

Accordingly, the graft containment cap 150 can be engaged with the head 120 of the graft retention tack 100 and slid into the head 120 of the graft retention tack 100 until each tooth 174 of the graft containment cap 150 snaps in place within each slot 132 in the head 120 of the graft retention tack 100 and engages each detent 138 within each slot 132.

In a particular embodiment, the graft retention tack 100 can be made from one or more resorbable materials. For example, the resorbable materials can include resorbable polymers, resorbable ceramics, or a combination thereof. In a particular embodiment, the resorbable polymers can include polylactide (PLA), polyglycolide (PGA), polylactide-co-glycolide (PLG), Poly-e-caprolactone, polydiaoxanone, polyanhydride, trimethylene carbonate, poly-β-hydroxybutyrate (PHB), poly-g-ethyl glutamate, poly-DTH-iminocarbonate, poly-bisphenol-A-iminocarbonate), polyorthoester (POE), polyglycolic lactic acid (PGLA), or a combination thereof. The resorbable ceramics can include calcium phosphate, calcium sulfate, calcium carbonate, or a combination thereof. The graft retention tack 100 can be made from other ceramics such as calcium sulfate, tricalcium phosphate, hydroxyapatite, or a combination thereof. Further, the graft retention tack 100 may be formed with or without a plasma coating. The coating may be a partial coating or a complete coating.

Further, in another embodiment, one or more of the materials described herein may be used as a substrate to receive another of the materials described herein. Further, the graft retention tack 100 may be partially porous, wholly porous, semi-porous, or non-porous. Also, the graft retention tack 100 can be may of an electrically conductive material, with or without one or more sensors incorporated therein, and can employ the use of one or more surfactants, wetting agents, hydrophilic coatings, hydrophobic coatings, or a combination thereof.

In order to manage electrically conductive surfaces of the graft retention tack 100, one or more insulating materials may be used to coat a portion or all of the graft retention tack 100. For example, the insulating materials can include parylene, polyimide, a combination thereof, or some other suitable insulating materials. The insulating materials can be layers and can be applied using vapor deposition, dip coating, light curing, etc. Any of the coatings described herein may be permanent, temporary, resorbable, or non-resorbable.

In a particular embodiment, a first carrier (not shown) can be placed within the post 102 of the graft retention tack 100. The first carrier can be wetted, or irrigated, with a bone growth factor that can promote bone growth. In this context, the term carrier can include a material or component that can absorb, adsorb or otherwise maintain a desired amount of a biological component, such as a growth factor, in the graft retention tack. The first carrier can be wetted before or after being placed within the post 102 of the graft retention tack 100. In this context, a bone growth factor can include any material generally suited to promote the growth, formation or viability of bone tissue. In a particular embodiment, the bone growth factor can include a relatively high dose of bone morphogenetic protein (BMP), e.g., BMP-2. For example, the BMP-2 can be mixed with sterile water in a range of about one milligram per milliliter to about five milligrams per milliliter (1.0 mg/ml to 5.0 mg/ml).

The bone growth factor can also include platelet derived growth factor (PDGF), insulin-like growth factor (IGF), LIM mineralization protein, fibroblast growth factor (FGF), osteoblast growth factor, growth and differentiation factor (GDF), vascular endothelial growth factor (VEGF), transformation growth factor beta (TGF-beta), or a combination thereof.

After the graft retention tack 100 is inserted in a patient, as described herein, the bone growth factor can seep from the graft retention tack 100 through the fenestrations 114 in the post 102 and can promote bone growth in the area around the post 102 of the graft retention tack 100.

Also, in a particular embodiment, a second carrier (not shown) can be placed within the head 120 of the graft retention tack 100 and the graft containment cap 150 can be fitted into the head 120 of the graft retention tack 100 in order to maintain the first carrier and the second carrier within the graft retention tack 1200. The second carrier can be wetted, or irrigated, with a cartilage growth factor that can promote cartilage growth. The second carrier can be wetted before or after being placed within the head 120 of the graft retention tack 100. In this context, a cartilage growth factor can include any material generally suited to promote the growth, formation or viability of cartilage tissue. In a particular embodiment, the cartilage growth factor can include a relatively low dose of bone morphogenetic protein, e.g., BMP-2. For example, the BMP-2 can be mixed with sterile water in a range of about one-tenths milligrams per milliliter to about five-tenths milligrams per milliliter (0.1 mg/ml to 0.5 mg/ml).

The cartilage growth factor can also include cartilage-derived morphogenetic protein (CDMP), platelet derived growth factor (PDGF), insulin-like growth factor (IGF), LIM mineralization protein, fibroblast growth factor (FGF), osteoblast growth factor, growth and differentiation factor (GDF), vascular endothelial growth factor (VEGF), transformation growth factor beta (TGF-beta), or a combination thereof.

After the graft retention tack 100 is inserted in a patient, as described herein, the cartilage growth factor can seep from the graft retention tack 100 through the holes in the head 120 of the graft retention tack 100, or between the cap engagement structures 124, 126, 128, 130 of the head 120, and the cartilage growth factor can promote cartilage growth in the area around the head 120 of the graft retention tack 100.

In a particular embodiment, each of the first carrier and the second carrier can be a collagen sponge, such as an absorbable collagen sponge. Further, each collagen sponge can include allogenic collagen, xenogenic collagen, autogenic collagen, recombinant collagen, or a combination thereof.

### Description of a First Method of Using a Graft Retention Tack

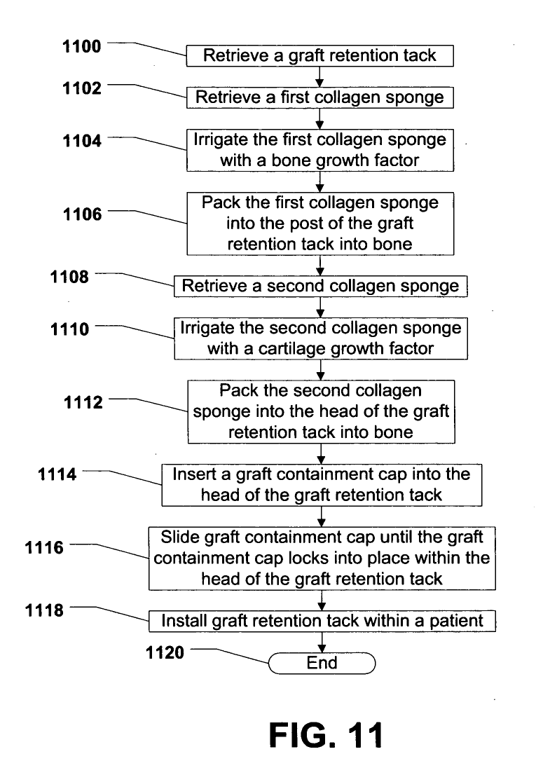

Referring now to FIG. 11, a method of installing a graft retention tack is shown and commences at block 1100. Commencing at block 1100, a graft retention tack can be retrieved. In a particular embodiment, the graft retention tack can be the graft retention tack described herein. Further, the graft retention tack can include a post, a head, and a graft containment cap.

Moving to block 1102, a first carrier can be retrieved. In a particular embodiment, the first collagen sponge can be an absorbable collagen sponge. At block 1104, the first collagen sponge can be irrigated with a bone growth factor, e.g., a bone growth factor described herein. Thereafter, at block 1106, the first collagen sponge can be packed, or otherwise inserted, in the post of the graft retention tack.

Continuing to block 1108, a second carrier can be retrieved. The second carrier can be an absorbable collagen sponge. At block 1110, the second collagen sponge can be irrigated with a cartilage growth factor, e.g., a cartilage growth factor described herein. Further, at block 1112, the second collagen sponge can be packed, or otherwise inserted, into the head of the graft retention tack.

At block 1114, a graft containment cap can be inserted into the head of the graft retention tack. Proceeding to block 1116, the graft containment cap can be slid into the head until the graft containment cap locks into place within the head of the graft retention tack. Thereafter, at block 1118, the graft retention tack can be installed in a patient. Then, the method ends at state 1120. In a particular embodiment, when the graft retention tack is installed in a patient, the post of the graft retention tack can be adjacent to, and in contact with, bone. Also, the head of the graft retention tack can be adjacent to, and in contact with, cartilage.

### Description of a Second Embodiment of a Graft Retention Tack

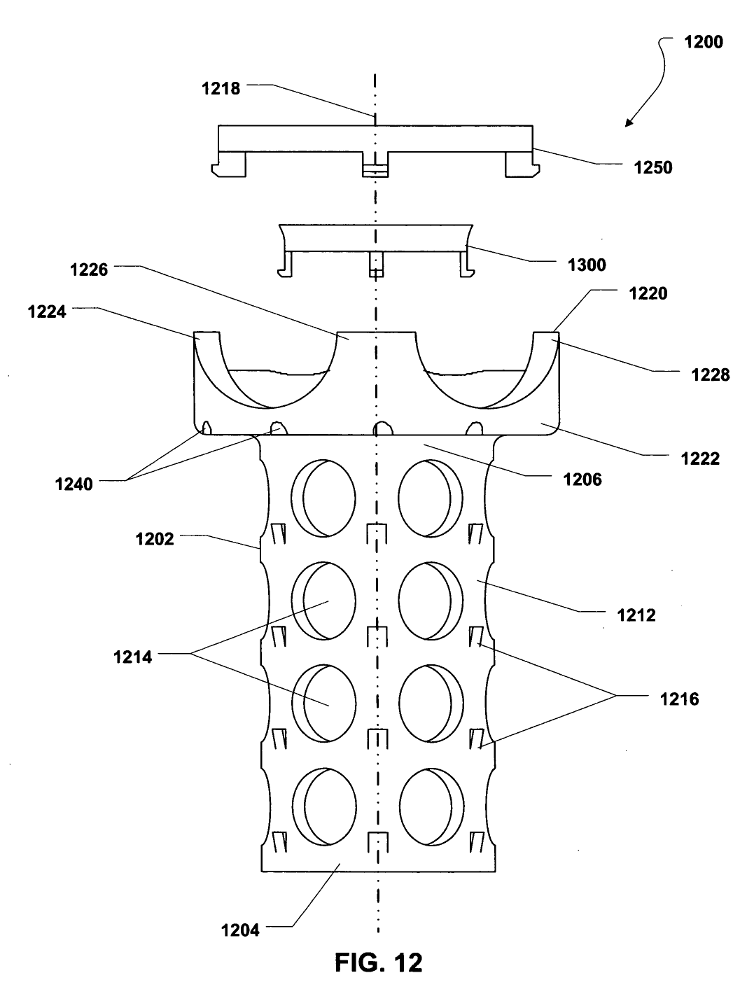

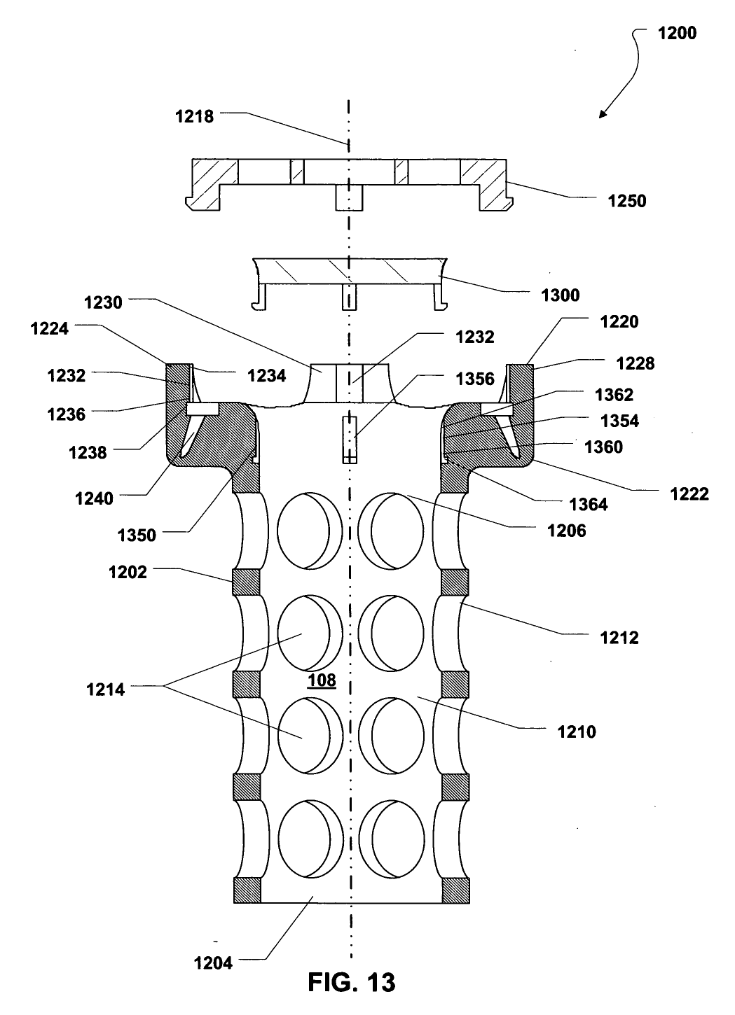

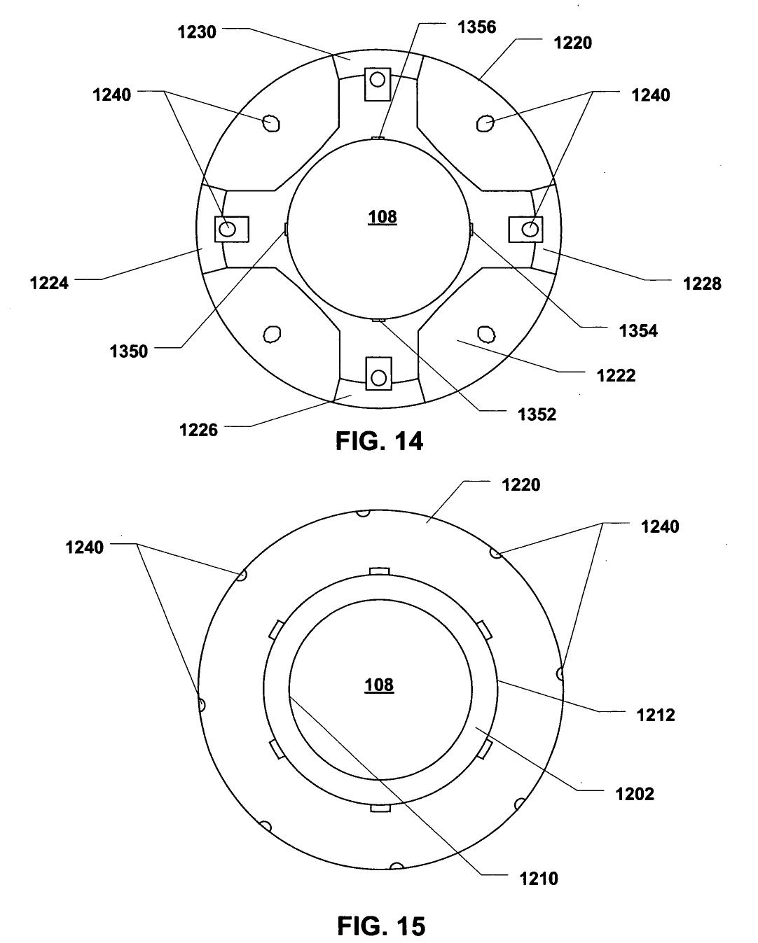

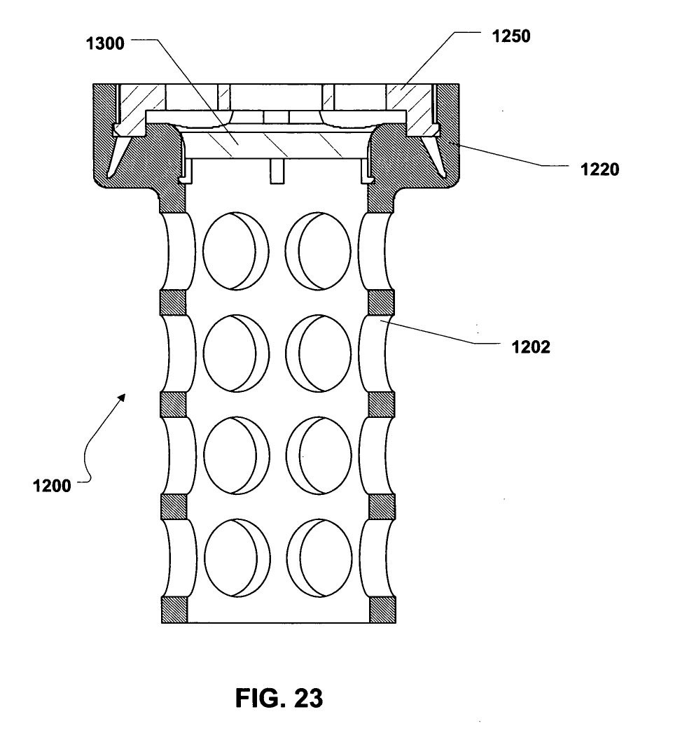

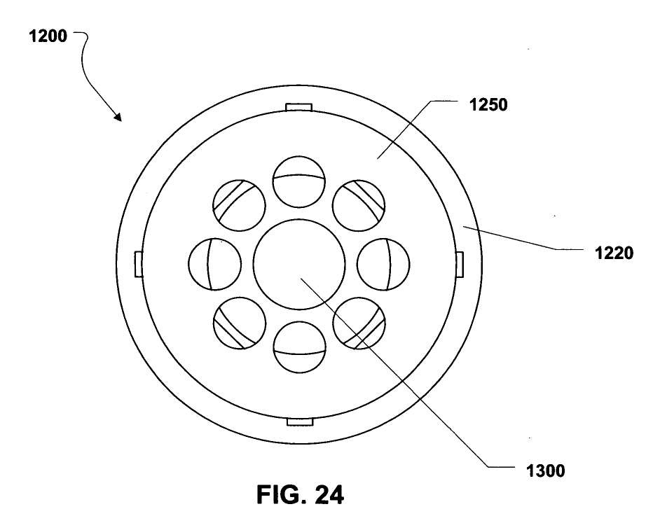

Referring to FIG. 12 through FIG. 24, a second embodiment of a graft retention tack is shown and is generally designated 1200. As illustrated in FIG. 12 and FIG. 13, the graft retention tack 1200 can include a post 1202. The post 1202 can include a proximal end 1204 and a distal end 1206. In a particular embodiment, the post 1202 can be generally cylindrical. Further, the post 1202 can be generally hollow and can include an interior chamber 1208. The post 1202 can also include an interior surface 1210 and an exterior surface 1212.

In a particular embodiment, the post 1202 of the graft retention tack 1200 can include a plurality of fenestrations 1214 formed in the post 1202 and leading from the interior chamber 1208 of the post 1202. Further, the post 1202 can include a plurality of tissue engagement structures 1216 that can extend radially from the exterior surface 1212 of the body 1202. The post 1202 can also define a longitudinal axis 1218. In an alternative embodiment, the post 1202 can be porous and the porosity of the post can allow a bone growth factor, described in detail below, to seep from the interior chamber 1208 of the post 1202. Further, the post 1202 itself can be dosed, or wetted, with a bone growth factor such that the bone growth factor is introduced into the pores of the post 1202. After installation, the bone growth factor can seep from the pores of the post 1202 of the graft retention tack 1200.

FIG. 12 and FIG. 13 further indicate that a head 1220 can be attached to the distal end 1206 of the post 1202. The head 1220 can be generally cylindrical and can include a base 1222 that can be oriented substantially perpendicular to the post 1202. A first cap engagement structure 1224 can extend substantially perpendicular from the base 1222 of the head. A second cap engagement structure 1226 can extend substantially perpendicular from the base 1222 of the head. A third cap engagement structure 1228 can extend substantially perpendicular from the base 1222 of the head. Moreover, a fourth cap engagement structure 1230 can extend substantially perpendicular from the base 1222 of the head 1220. Each cap engagement structure 1224, 1226, 1228, 1230 can be substantially parallel to the longitudinal axis 1218 of the post 1202.

As illustrated in FIG. 13, each cap engagement structure 1224, 1226, 1228, 1230 can include a slot 1232. Each slot 1232 can include a proximal end 1234 and a distal end 1236. Further, the proximal end 1234 of each slot 1232 can be formed with a detent 1238. As described in detail below, the detent 1238 of each slot 1232 can be configured to receive and engage a locking arm from a graft containment cap.

FIG. 13 through FIG. 15 further show that the head 1220 of the graft retention tack 1200 can include a plurality of holes 1240 therethrough. In a particular embodiment, the holes 1240 can be formed in the base 1222 of the head 1220. Further, the holes 1240 can be equally spaced radially around the base 1222 of the head 1220. Also, the holes 1240 can be formed at an angle through the base 1222 of the head 1220.

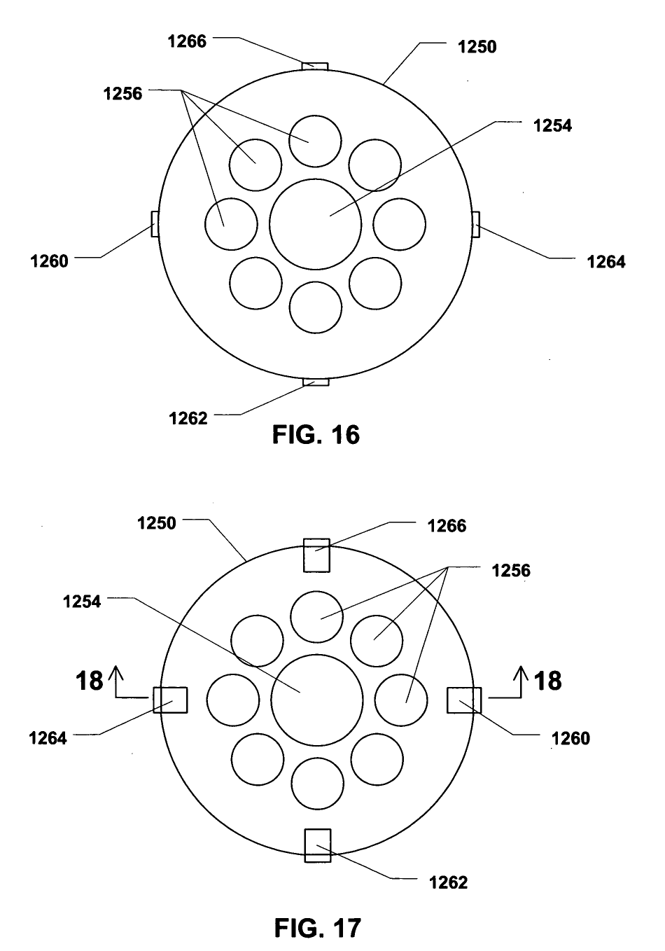

As shown in FIG. 12 and FIG. 13, the graft retention tack 1200 can further include an exterior graft containment cap 1250. As illustrated in FIG. 16 through FIG. 18, the exterior graft containment cap 1250 can include a base 1252. In a particular embodiment, the base 1252 can be a generally cylindrical disc. The base 1252 can be formed with a central hole 1254 therethrough. Further, the base 1252 can be formed with a plurality of radial holes 1256 that can be equally spaced radially around the central hole 1254 within the base 1252.

FIG. 16 through FIG. 18 indicate that the exterior graft containment cap 1250 can include a first locking arm 1260 that can extend substantially perpendicular from the base 1252 of the exterior graft containment cap 1250. A second locking arm 1262 can extend substantially perpendicular from the base 1252 of the exterior graft containment cap 1250. A third locking arm 1264 can also extend substantially perpendicular from the base 1252 of the exterior graft containment cap 1250. Further, a fourth locking arm 1266 can extend substantially perpendicular from the base 1252 of the exterior graft containment cap 1250.

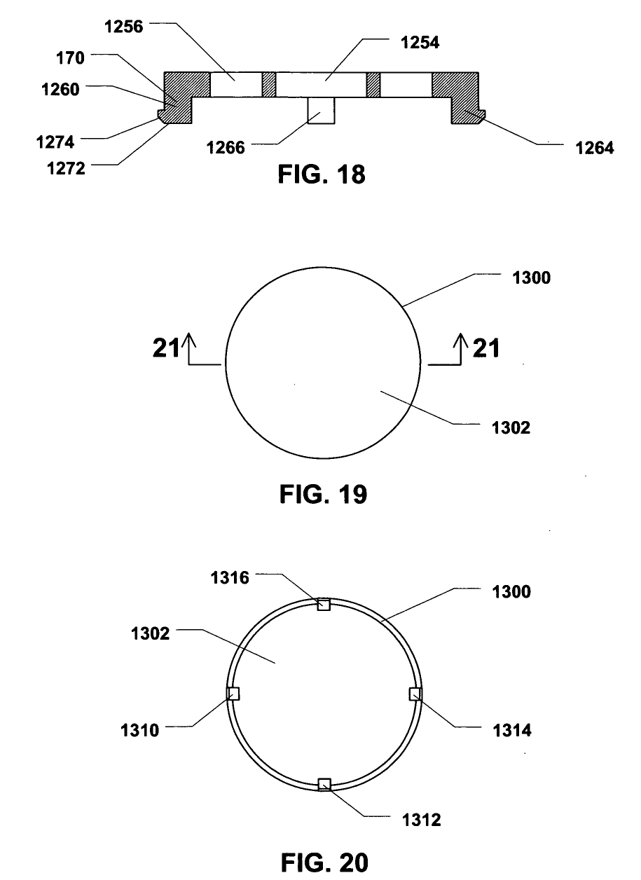

As illustrated in FIG. 18, each locking arm 1260, 1262, 1264, 1266 can include a proximal end 1270 and a distal end 1272. Further, a tooth 1274 can extend from the distal end 1272 of each locking arm 1260, 1262, 1264, 1266. In a particular embodiment, the exterior graft containment cap 1250 can fit into the head 1220 of the graft retention tack 1200. Further, each locking arm 1260, 1262, 1264, 1266 can fit into a cap engagement structure 1224, 1226, 1228, 1230. In particular, each locking arm 1260, 1262, 1264, 1266 can fit into a slot 1232 formed in a cap engagement structure 1224, 1226, 1228, 1230. Moreover, the tooth 1274 that extends from each locking arm 1260, 1262, 1264, 1266 can engage the detent 1238 formed in the slot 1232 of each cap engagement structure 1224, 1226, 1228, 1230.

Accordingly, the exterior graft containment cap 1250 can be engaged with the head 1220 of the graft retention tack 1200 and slid into the head 1220 of the graft retention tack 1200 until each tooth 1274 of the exterior graft containment cap 1250 snaps in place within each slot 1232 in the head 1220 of the graft retention tack 1200 and engages each detent 1238 within each slot 1232.

FIG. 12 and FIG. 13 also indicate that the graft retention tack 1200 can include an interior graft containment cap 1300. The interior graft containment cap 1300 can include a base 1302. FIG. 19 through FIG. 21 indicate that the interior graft containment cap 1300 can include a first locking arm 1310 that can extend substantially perpendicular from the base 1302 of the interior graft containment cap 1300. A second locking arm 1312 can extend substantially perpendicular from the base 1302 of the interior graft containment cap 1300. A third locking arm 1314 can also extend substantially perpendicular from the base 1302 of the interior graft containment cap 1300. Further, a fourth locking arm 1316 can extend substantially perpendicular from the base 1302 of the interior graft containment cap 1300.

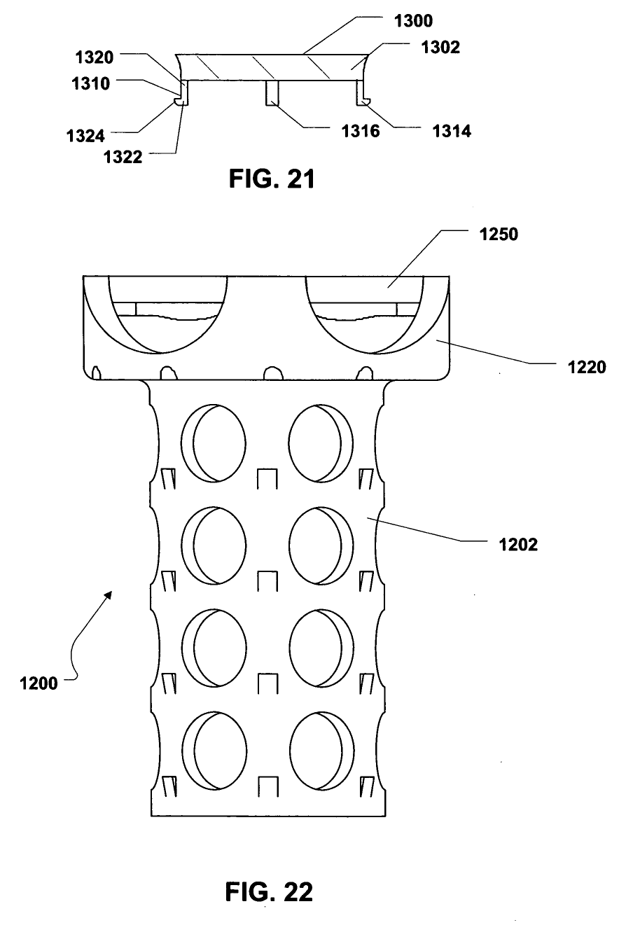

As illustrated in FIG. 21, each locking arm 1310, 1312, 1314, 1316 can include a proximal end 1320 and a distal end 1322. Further, a tooth 1324 can extend from the distal end 1322 of each locking arm 1310, 1312, 1314, 1316.

In a particular embodiment, the interior graft containment cap 1300 can fit into the distal end 1206 of the post 1202 of the graft retention tack 1200. Specifically, as illustrated in FIG. 13, the distal end 1206 of the post 120 of the graft retention tack 1200 can include a first slot 1350, a second slot 1352, a third slot 1354, and a fourth slot 1356. Each slot 1350, 1352, 1354, 1356 can include a proximal end 1360 and a distal end 1362. Further, the proximal end 1360 of each slot 1350, 1352, 1354, 1356 can be formed with a detent 1364. As described in detail below, the detent 1364 of each slot 1350, 1352, 1354, 1356 can be configured to receive and engage a corresponding locking arm 1310, 1312, 1314, 1316 from the interior graft containment cap 1300.

In particular, each locking arm 1310, 1312, 1314, 1316 of the interior graft containment cap 1300 can fit into a corresponding slot 1350, 1352, 1354, 1356 formed in the distal end 1206 of the post 1202 of the graft retention tack 1200. Moreover, the tooth 1364 that extends from each locking arm 1310, 1312, 1314, 1316 can engage the detent 1364 formed in the corresponding slot 1350, 1352, 1354, 1356 of the post 1202.

Accordingly, the interior graft containment cap 1300 can be engaged with the distal end 1206 of the post 1202 of the graft retention tack 1200 and slid into the distal end 1206 of the post 1202 of the graft retention tack 1200 until each tooth 1364 of the interior graft containment cap 1300 snaps in place within each slot 1350, 1352, 1354, 1356 in the post 1202 of the graft retention tack 1200 and engages each detent 1364 within each slot 1350, 1352, 1354, 1356.

In a particular embodiment, the graft retention tack 1200 can be made from one or more resorbable materials. For example, the resorbable materials can include resorbable polymers, resorbable ceramics, or a combination thereof. In a particular embodiment, the resorbable polymers can include polylactide (PLA), polyglycolide (PGA), polylactide-co-glycolide (PLG), Poly-e-caprolactone, polydiaoxanone, polyanhydride, trimethylene carbonate, poly-β-hydroxybutyrate (PHB), poly-g-ethyl glutamate, poly-DTH-iminocarbonate, poly-bisphenol-A-iminocarbonate), polyorthoester (POE), polyglycolic lactic acid (PGLA), or a combination thereof. The resorbable ceramics can include calcium phosphate, calcium sulfate, calcium carbonate, or a combination thereof. The graft retention tack 100 can be made from other ceramics such as calcium sulfate, tricalcium phosphate, hydroxyapatite, or a combination thereof. Further, the graft retention tack 1200 may be formed with or without a plasma coating. The coating may be a partial coating or a complete coating.

Further, in another embodiment, one or more of the materials described herein may be used as a substrate to receive another of the materials described herein. Further, the graft retention tack 1200 may be partially porous, wholly porous, semi-porous, or non-porous. Also, the graft retention tack 1200 can be may of an electrically conductive material, with or without one or more sensors incorporated therein, and can employ the use of one or more surfactants, wetting agents, hydrophilic coatings, hydrophobic coatings, or a combination thereof.

In order to manage electrically conductive surfaces of the graft retention tack 1200, one or more insulating materials may be used to coat a portion or all of the graft retention tack 1200. For example, the insulating materials can include parylene, polyimide, a combination thereof, or some other suitable insulating materials. The insulating materials can be layers and can be applied using vapor deposition, dip coating, light curing, etc. Any of the coatings described herein may be permanent, temporary, resorbable, or non-resorbable.

In a particular embodiment, a first carrier (not shown) can be placed within the post 1202 of the graft retention tack 1200 and the interior graft retention cap 1300 can be inserted into the distal end 1206 of the post 1202 of the graft retention tack 1200 in order to substantially seal the distal end 1206 of the post 1202 of the graft retention tack 1200. The first carrier can be wetted, or irrigated, with a bone growth factor that can promote bone growth. In this context, the term carrier can include a material or component that can absorb, adsorb or otherwise maintain a desired amount of a biological component, such as a growth factor, in the graft retention tack. The first carrier can be wetted before or after being placed within the post 1202 of the graft retention tack 1200. In this context, a bone growth factor can include any material generally suited to promote the growth, formation or viability of bone tissue. In a particular embodiment, the bone growth factor can include a relatively high dose of bone morphogenetic protein, e.g., BMP-2. For example, the BMP-2 can be mixed with sterile water in a range of about one milligram per milliliter to about five milligrams per milliliter (1.0 mg/ml to 5.0 mg/ml).

The bone growth factor can also include platelet derived growth factor (PDGF), insulin-like growth factor (IGF), LIM mineralization protein, fibroblast growth factor (FGF), osteoblast growth factor, growth and differentiation factor (GDF), vascular endothelial growth factor (VEGF), transformation growth factor beta (TGF-beta), or a combination thereof.

After the graft retention tack 1200 is inserted in a patient, as described herein, the bone growth factor can seep from the graft retention tack 1200 through the fenestrations 1214 in the post 1202 and can promote bone growth in the area around the post 1202 of the graft retention tack 1200.

Also, in a particular embodiment, a second carrier (not shown) can be placed within the head 1220 of the graft retention tack 1200 and the exterior graft containment cap 1250 can be fitted into the head 1220 of the graft retention tack 1200 in order to maintain the second carrier within the head 1220 of the graft retention tack 1200. The second carrier can be wetted, or irrigated, with a cartilage growth factor that can promote cartilage growth. The second carrier can be wetted before or after being placed within the head 1220 of the graft retention tack 1200. In this context, a cartilage growth factor can include any material generally suited to promote the growth, formation or viability of cartilage tissue. In a particular embodiment, the cartilage growth factor can include a relatively low dose of bone morphogenetic protein, e.g., BMP-2. For example, the BMP-2 can be mixed with sterile water in a range of about one-tenths milligrams per milliliter to about five-tenths milligrams per milliliter (0.1 mg/ml to 0.5 mg/ml).

The cartilage growth factor can also include cartilage-derived morphogenetic protein (CDMP), platelet derived growth factor (PDGF), insulin-like growth factor (IGF), LIM mineralization protein, fibroblast growth factor (FGF), osteoblast growth factor, growth and differentiation factor (GDF), vascular endothelial growth factor (VEGF), transformation growth factor beta (TGF-beta), or a combination thereof.

After the graft retention tack 1200 is inserted in a patient, as described herein, the cartilage growth factor can seep from the graft retention tack 1200 through the holes in the head 1220 of the graft retention tack 1200, or between the cap engagement structures 1224, 1226, 1228, 1230 of the head 1220, and the cartilage growth factor can promote cartilage growth in the area around the head 1220 of the graft retention tack 1200.

In a particular embodiment, each of the first carrier and the second carrier can be a collagen sponge, such as an absorbable collagen sponge. Further, each collagen sponge can include allogenic collagen, xenogenic collagen, autogenic collagen, recombinant collagen, or a combination thereof.

### Description of a Second Method of Using a Graft Retention Tack

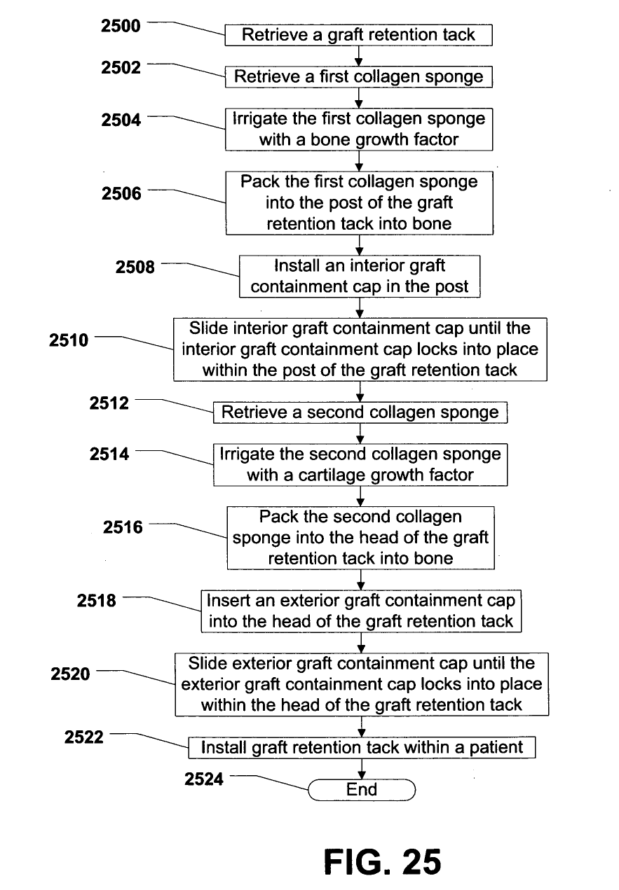

Referring now to FIG. 25, a method of installing a graft retention tack is shown and commences at block 2500. Commencing at block 2500, a graft retention tack can be retrieved. In a particular embodiment, the graft retention tack can be a graft retention tack according to one or more of the embodiments described herein. Further, the graft retention tack can include a post, a head, an interior graft containment cap, and an exterior graft containment cap.

Moving to block 2502, a first carrier can be retrieved. In a particular embodiment, the first carrier can be an absorbable collagen sponge. At block 2504, the first collagen sponge can be irrigated with a bone growth factor, e.g., a bone growth factor described herein. Thereafter, at block 2506, the first collagen sponge can be packed, or otherwise inserted, in the post of the graft retention tack.

Proceeding to block 2508, the interior graft containment cap can be inserted into the distal end of the post of the graft retention tack. Proceeding to block 2510, the interior graft containment cap can be slid into the distal end of the post until the interior graft containment cap locks into place within the distal end of the post of the graft retention tack.

Continuing to block 2512, a second carrier can be retrieved. The second carrier can be an absorbable collagen sponge. At block 2514, the second collagen sponge can be irrigated with a cartilage growth factor, e.g., a cartilage growth factor described herein. Further, at block 2516, the second collagen sponge can be packed, or otherwise inserted, into the head of the graft retention tack.

At block 2518, an exterior graft containment cap can be inserted into the head of the graft retention tack. Proceeding to block 2520, the exterior graft containment cap can be slid into the head until the exterior graft containment cap locks into place within the head of the graft retention tack. Thereafter, at block 2522, the graft retention tack can be installed in a patient. Then, the method ends at state 2524. In a particular embodiment, when the graft retention tack is installed in a patient, the post of the graft retention tack can be adjacent to, and in contact with, bone. Also, the head of the graft retention tack can be adjacent to, and in contact with, cartilage.

### Conclusion

With the configuration of structure described above, the graft retention tack provides a device that can be used to contain allogenic or synthetic graft materials for inducing bone or tissue growth or regeneration. Specifically, the graft retention tack can contain a first absorbable collagen sponge irrigated with a bone growth factor for promoting bone growth and a second absorbable collagen sponge irrigated with a cartilage growth factor for promoting cartilage growth. As such, when placed in a patient, the graft retention tack can promote bone growth around a post of the graft retention tack in which the first absorbable collagen sponge can be inserted. Also, the graft retention tack can promote cartilage growth around a head of the graft retention tack in which the second absorbable collagen sponge can be inserted.

Further, the graft retention tack can be made from a resorbable material. Accordingly, as bone or tissue grows around the graft retention tack, the graft retention tack can be resorbed by the human body in which the graft retention tack is installed.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

1. A graft retention tack, comprising:
a post configured to receive a bone growth factor; and
a head configured to receive a cartilage growth factor.

2. The graft retention tack of claim 1, wherein the graft retention tack is configured to be installed within a patient such the post is in contact with bone and the head is in contact with cartilage.

3. The graft retention tack of claim 1, wherein the bone growth factor comprises bone morphogenetic protein (BMP) mixed with sterile water in a range of about one milligram per milliliter to about five milligrams per milliliter (1.0 mg/ml to 5.0 mg/ml).

4. The graft retention tack of claim 1, wherein the bone growth factor comprises BMP-2, platelet derived growth factor (PDGF), insulin-like growth factor (IGF), LIM mineralization protein, fibroblast growth factor (FGF), osteoblast growth factor, growth and differentiation factor (GDF), vascular endothelial growth factor (VEGF), transformation growth factor beta (TGF-beta), or a combination thereof.

5. The graft retention tack of claim 1, wherein the cartilage growth factor comprises BMP-2 mixed with sterile water in a range of about one-tenths milligrams per milliliter to about five-tenths milligrams per milliliter (0.1 mg/ml to 0.5 mg/ml).

6. The graft retention tack of claim 1, wherein the cartilage growth factor comprises BMP-2, cartilage-derived morphogenetic protein (CDMP), platelet derived growth factor (PDGF), insulin-like growth factor (IGF), LIM mineralization protein, fibroblast growth factor (FGF), osteoblast growth factor, growth and differentiation factor (GDF), vascular endothelial growth factor (VEGF), transformation growth factor beta (TGF-beta), or a combination thereof.

7. The graft retention tack of claim 1, wherein the graft retention tack is formed of a resorbable material.

8. The graft retention tack of claim 7, wherein the resorbable material comprises a resorbable polymer, a resorbable ceramic, or a combination thereof.

9. The graft retention tack of claim 8, wherein the resorbable polymer comprises polylactide (PLA), polyglycolide (PGA), polylactide-co-glycolide (PLG), Poly-e-caprolactone, polydiaoxanone, polyanhydride, trimethylene carbonate, poly-β-hydroxybutyrate (PHB), poly-g-ethyl glutamate, poly-DTH-iminocarbonate, poly-bisphenol-A-iminocarbonate), polyorthoester (POE), polyglycolic lactic acid (PGLA), or a combination thereof.

10. The graft retention tack of claim 8, wherein the resorbable ceramic comprises calcium phosphate, calcium sulfate, calcium carbonate, or a combination thereof.

11. The graft retention tack of claim 1, wherein the post comprises:
a plurality of fenestrations leading from an interior chamber of the post wherein the fenestrations are configured to allow the bone growth factor to exit the post.

12. The graft retention tack of claim 1, further comprising a graft containment cap configured to be installed within the head of the graft retention tack.

13. The graft retention tack of claim 12, wherein the graft containment cap includes a plurality of holes configured to allow the cartilage growth factor to exit the head.

14. The graft retention tack of claim 1, wherein the head includes a plurality of holes configured to allow the cartilage growth factor to exit the head.

15. The graft retention tack of claim 12, wherein the head of the graft retention tack comprises:
at least one cap engagement structure;
a slot formed in the at least one cap engagement structure; and
a detent formed at an end of the slot.

16. The graft retention tack of claim 15, wherein the graft containment cap comprises:
a generally disc shaped base;
at least one locking arm extending from the base; and
a tooth extending from the at least one locking arm.

17. The graft retention tack of claim 16, wherein the at least one locking arm of the graft containment cap is configured to fit into the slot formed in the at least one cap engagement structure.

18. The graft retention tack of claim 17, wherein the tooth extending from the at least one locking arm is configured to engage the detent formed at the end of the slot in the cap engagement structure and lock the graft containment cap within the head of the graft retention tack.

19. A graft retention tack, comprising:
a post having a proximal end and a distal end, wherein the post is configured to receive a first carrier; and
a head attached to the distal end of the post, wherein the head is configured to receive a second carrier.

20. (canceled)

21. (canceled)

22. The graft retention tack of claim 19, further comprising an interior graft containment cap configured to fit into the distal end of the post.

23. The graft retention tack of claim 22, wherein the interior graft containment cap substantially seals the distal end of the post.

24. The graft retention tack of claim 22, further comprising an exterior graft containment cap configured to fit into the head.

25. The graft retention tack of claim 24, wherein the exterior graft containment cap substantially maintains the second carrier within the head.

26. A method of treating a patient using a graft retention tack having a post and a head attached to the post, comprising:
wetting a first carrier with a first growth factor;
inserting the first carrier into a post of the graft retention tack;
wetting a second carrier with a second growth factor; and
inserting the second carrier into a head of the graft retention tack.

27. The method of claim 26, further comprising:
engaging a graft containment cap with the head of the graft retention tack.

28. The method of claim 27, further comprising:
inserting the graft retention tack in a patient so that the post is at least partially engaged with bone and the head is at least partially engaged with cartilage.

29. (canceled)

30. (canceled)

31. (canceled)

32. A method of treating a patient using a graft retention tack having a post and a head attached to the post, comprising:
wetting a first carrier with a first growth factor;
inserting the first carrier into a post of the graft retention tack; and
engaging an interior graft retention cap with the post to substantially seal the post.

33. (canceled)

34. (canceled)

35. (canceled)

36. A method of treating a patient using a graft retention tack having a post and a head attached to the post, comprising:
inserting a first carrier into a post of the graft retention tack; and
inserting a second carrier into a head of the graft retention tack.

37. (canceled)

38. (canceled)

39. (canceled)

40. (canceled)
